(19) United States
(12) Patent Application Publication
(10) Pub. No.: US 2016/0158466 A1
(43) Pub. Date: Jun. 9, 2016

(54) REPLACEMENT CARTRIDGE

(71) Applicant: MEDIC ACTIV VERTRIEBS GMBH, Grünwald (DE)

(72) Inventors: Peter BRUNNER, Ismaning (DE); Bernd Heiko LINDENA, Westendorf (AT)

(21) Appl. No.: 14/907,240

(22) PCT Filed: Jul. 23, 2014
(86) PCT No.: PCT/EP2014/065859

§ 371 Date: Jan. 22, 2016

(30) Foreign Application Priority Data
Jul. 24, 2013 (DE) 10 2013 214 471.7

(51) Int. Cl. A61M 11/02; A61L 9/14

(52) U.S. Cl. CPC A61M 11/02; A61L 9/14; A61M 2202/0208

(57) ABSTRACT

Embodiments of the present invention relate to an exchangeable cartridge for generating an aerosol with at least one bottom element and with at least one cover element, wherein in the bottom element and/or the cover element a reservoir is formed for a first, in particular liquid, aerosol component, wherein in the exchangeable cartridge a nebulizer nozzle is formed, wherein the bottom element and the cover element are connected to each other by a bayonet catch.

### CROSS-REFERENCE TO RELATED APPLICATION

This application is a National Phase Patent Application of International Patent Application Number PCT/EP2014/065859, filed on Jul. 23, 2014, which claims priority to and the benefit of German Patent Application No. 10 2013 214 471.7 filed 24 Jul. 2013, the disclosure of which is hereby incorporated herein by reference.

### TECHNICAL FIELD

Embodiments of the present invention relate to an exchangeable cartridge for generating an aerosol, with at least one bottom element and at least one cover element, wherein in the bottom element and/or the cover element a reservoir is formed for a first, in particular liquid, aerosol component.

### TECHNOLOGICAL BACKGROUND

Exchangeable cartridges of the above mentioned type are, amongst others, used in atomizers, inhalers or cabins, to expose the respiratory tracts or bodies of living beings to an aerosol. This may be carried out for therapeutic purposes or for disinfection purposes.

In WO 2011/082 838 an exchangeable cartridge for generating an aerosol is described, which consists of at least two components which are connectable to each other after filling in a fluid to be nebulized. This may be carried out via a plug connection or a screw connection.

For establishing a screw connection, the components have to be accurately aligned to each other and in this position rotated with respect to each other. This results in an increased time-effort when closing the cartridge after filling in the fluid to be nebulized. In addition, the process in view of large piece numbers is poorly automatable. A simple plug connection offers only little protection against unintentionally opening the cartridge.

Therefore, according to WO 2011/082 838, a non-detachable connection of the both components by adhering and in particular by welding is preferred. It has turned out that after a longer storage of adhered or welded cartridges, leakages may occur.

In DE 10 2011 079 810 an ampoule is described for inserting into an aerosol generator to atomize a substance contained in the ampoule. The ampoule comprises a container portion which forms a chamber for accommodating at least one constituent of the substance, and an interface portion which is formed for accommodating and fixing the ampoule in the accommodation portion of the aerosol generator by which aerosol generator then an aerosol is generated from the substance.

In DE 87 04 273 a device for spraying compounds is described, consisting of a pressure container with an opening which is pressure tightly closable by a cover, for introducing the compound to be sprayed into the container interior, with a spaying nozzle which is arranged outside of the container, which is connected to the container interior via a passage to be opened by a spraying valve, and with means for introducing a pressure medium into the container interior.

In DE 199 40 713 a cartridge for liquids is described. The cartridge is a three-shell container which may consist of a collapsible bag containing the liquid, a dimensionally stable container and a stiff metal sheath. The cartridge may be detachably connected to a removal device which serves as separate nebulizer for the generation of an inhalable aerosol for a treatment of diseases.

### SUMMARY OF THE INVENTION

Based on this, there may be a need for providing a cartridge of the above mentioned type, which is easier to manufacture and comprises an improved protection against leakages.

According to embodiments of the invention, an exchangeable cartridge is provided for generating an aerosol, with at least one bottom element and with at least one cover element, wherein in the bottom element and/or the cover element a reservoir is formed for a first, in particular liquid, aerosol component, wherein in the exchangeable cartridge a nebulizer nozzle is formed, and wherein the bottom element and the cover element are connected to each other by a bayonet catch.

An advantage of the above described exchangeable cartridge may be that the nebulizer nozzle is formed within the exchangeable cartridge, such that the nebulizer nozzle is protected against dirt and damage. Therefore, an even and safe nebulization may be achieved.

By the translational motion towards each other, the bottom element and the cover element may be aligned for the subsequent rotational motion in an easy manner. In addition, for closing a bayonet catch, only a time-saving small angle of rotation is required.

Furthermore, the use of a bayonet catch may, in contrast to a screw locking, allow for larger dimension tolerances when manufacturing the exchangeable cartridge. Thus, more cost-efficient production processes may be used, for example injection molding.

The bayonet catch may be formed by protrusions, for example, which are engaged by corresponding hooks. The selection of three pairs of protrusions and hooks for the bayonet catch may enable a smaller angle of rotation while at the same time unproblematically bringing together the bottom element and the cover element.

According to an embodiment, the bottom element and/or the cover element comprises a centering surface. The centering surface may simplify the assembling of the bottom element and the cover element, which is required before the rotational motion.

According to a further embodiment, the exchangeable cartridge comprises at least one engagement protrusion assigned to the bayonet catch and at least one engagement accommodation assigned to the bayonet catch, wherein the bottom element and the cover element are engageable with each other by the engagement protrusion and the engagement accommodation. By an engaging, a rotational motion which is sufficient for a safe and durable connection of the bottom element and the cover element can be recognized by locking persons or machines. In addition, by engaging, an unintentionally opening of the cartridge can be counteracted.

According to a further design, the engagement protrusion and/or the engagement accommodation are formed such that the bottom element and the cover element are engageable with each other only once. In this way, it can be avoided that the exchangeable cartridge can be opened by unauthorized persons and refilled with an unsuitable aerosol component.

Further, an embodiment of the exchangeable cartridge provides that the engagement protrusion and/or the engagement accommodation comprise a predetermined breaking location which, when opening the engagement, leads to a destruction, in particular a visible destruction, of the engagement protrusion and/or the engagement accommodation. By providing a predetermined breaking location, it may be enabled that the exchangeable cartridge can indeed be re-opened, but cannot be safely closed again. Opening the exchangeable cartridge may for example enable to remove remains of the second aerosol component after the use of the exchangeable cartridge and to deliver them to a waste which is separated from the other constituents of the exchangeable cartridge.

The predetermined breaking location may be provided in particular only at the bottom element and cover element, respectively. In this case, the undestroyed cover element and bottom element, respectively, may be re-used after a cleaning and/or sterilization, if necessary, and may in this way contribute to a sustainable consumption of resources. By the visibility of the destroyed predetermined breaking location after opening, it may be enabled for a user of the exchangeable cartridge to safely distinguish this from originally packaged exchangeable cartridges.

According to a further design, the exchangeable cartridge further comprises a sealing ring arranged between the bottom element and the cover element. With the sealing ring, a fluid tight inclusion of the second aerosol component within the exchangeable cartridge may be ensured. The sealing ring may in particular be arranged axially between the bottom element and the cover element, and may seal a front face of the bottom element and a front face of the cover element with respect to each other. Likewise, the sealing ring may be arranged radially between the bottom element and the cover element and may seal an outer circumferential surface of the bottom element and the cover element, respectively, with respect to an inner circumferential surface of the cover element and the bottom element, respectively, such that also at a low pressure exerted by the bayonet catch in an axial direction, a good sealing can be ensured. Likewise, the sealing ring may, before assembling the bottom element and the cover element, be slid secured against loss, onto the outer circumferential surface of the bottom element and the cover element, respectively.

Moreover, according to an embodiment, the exchangeable cartridge comprises a pressure gas bottle, in particular a pressure gas cartridge. An exchangeable cartridge which comprises a pressure gas bottle, in particular a pressure gas cartridge may be employed autarkicly. In particular, a separate compressor for generating a pressure gas for outputting the aerosol can be omitted. This may be reasonable in particular in moist environments, where electrical installations have to meet special safety standards.

The gas bottle may be provided with a valve, such that outputting the aerosol can be interrupted and continued later on. However, also a pressure bottle without a valve may be used, such that for each application an accurately defined amount of gas can be provided.

The pressure gas bottle may be detachably connected to the bottom element and/or the cover element. In this way, the type of the gas which is used for outputting the first aerosol component located in the reservoir can be exchanged in dependency from the intended application. For example, it may be intended to output the first aerosol component usually with cost-efficiently available nitrogen, and to use oxygen in special cases. Likewise it may be enabled to use the content of a pressure gas bottle with different first aerosol components. This may be in particular appropriate when different first aerosol components with the same type of gas are output. For establishing the detachable connection, the gas bottle can be screwed into the bottom element and/or the cover element, for example. This type of connection may have the advantage, that cost-efficiently available, customary pressure gas bottles can be used. In particular pressure gas bottles may be considered which usually are intended for inflating rescue means, i.e. life vests or rescue isles. Likewise, the connection may be established by a bayonet catch. The advantages accompanied with this type of connection have already been referred to above with respect to the connection of bottom element / cover element.

It is also possible to non-detachably connect the pressure gas bottle to the bottom element and/or the cover element. In this case, it can be ensured that always a certain combination of gas and first aerosol component are applied and the components are not mixed in an inadmissible, possibly harmful manner. Further, this design with the non-detachable connection may simplify the use of the exchangeable cartridge, due to the fact that only exchanging one single element, i.e. the exchangeable cartridge, can be sufficient.

Further, the design of the exchangeable cartridge provides that the pressure gas bottle contains a fragrant and/or disinfectant and/or therapeutically effective gas, in particular oxygen. In other words, the gas is not used for passively outputting and nebulizing the first aerosol component. Such a design may allow to perform an adjustment between the first aerosol component and the gas for achieving a high success in a therapy.

### DETAILED DESCRIPTION OF EXEMPLARY EMBODIMENTS

In the Figures shows

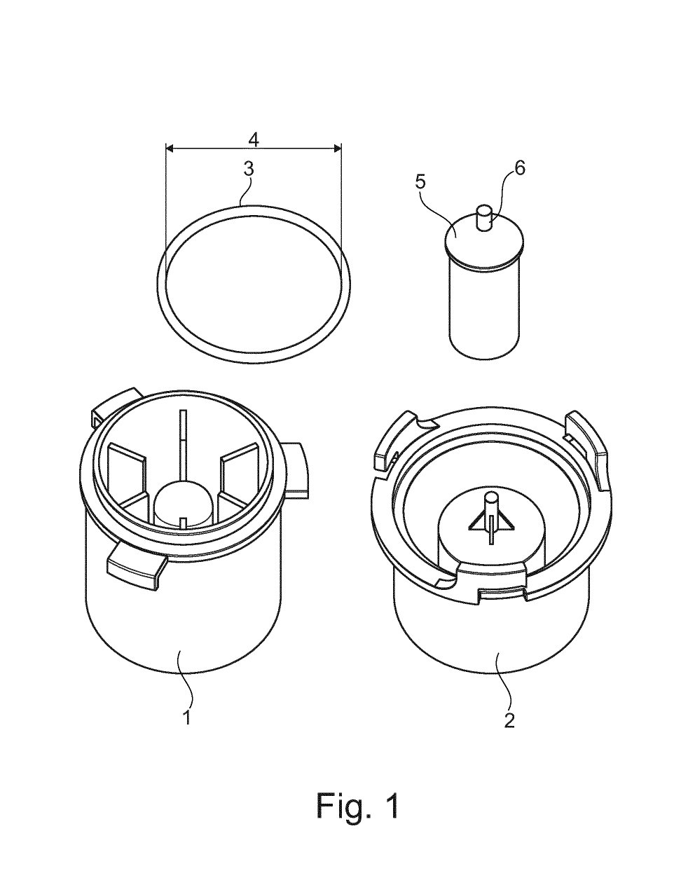

FIG. 1 a bottom element, a cover element, a sealing ring and a nozzle insert;

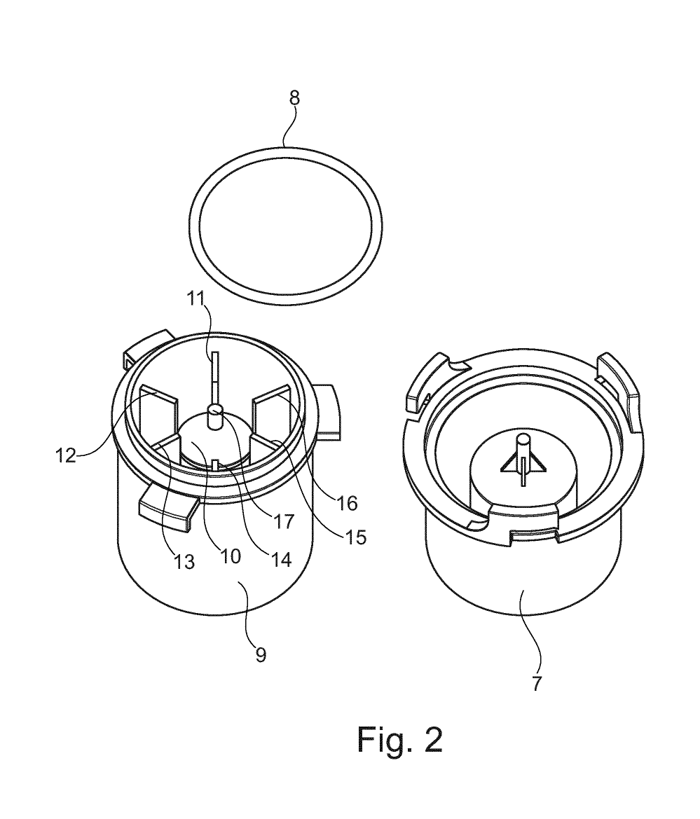

FIG. 2 a cover element, a sealing ring and a bottom element;

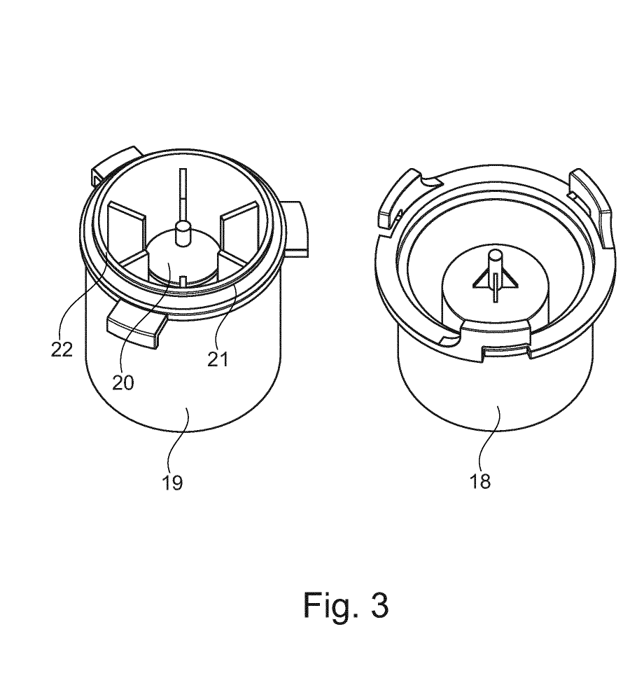

FIG. 3 a cover element and a bottom element;

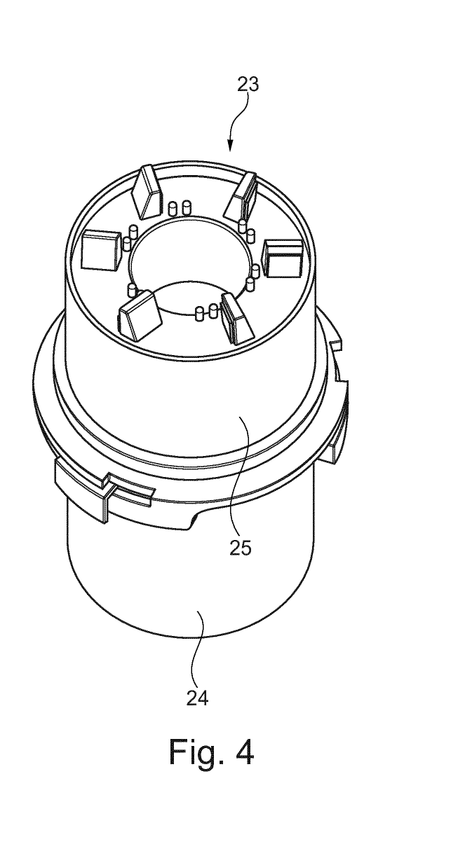

FIG. 4 an exchangeable cartridge;

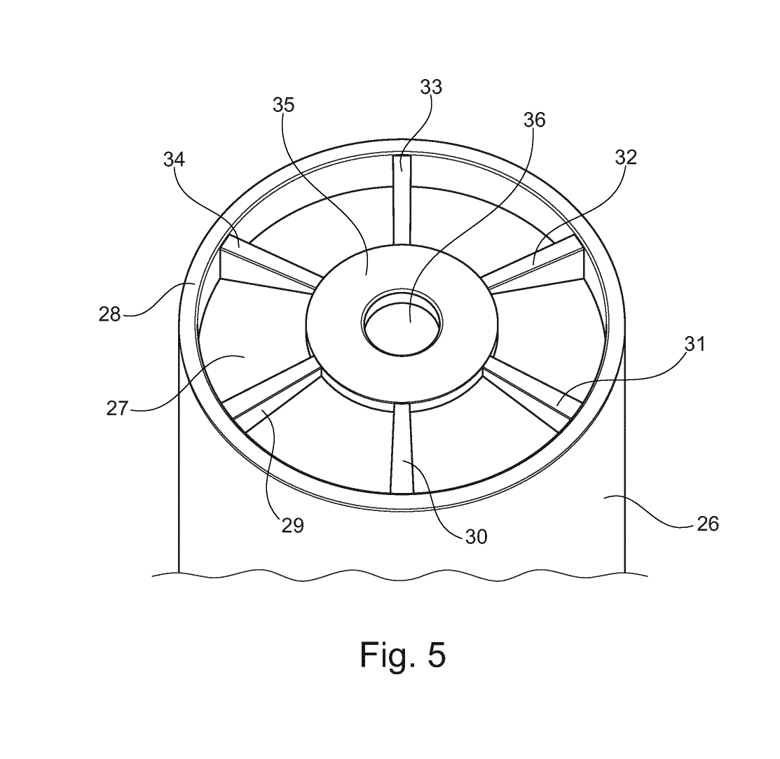

FIG. 5 the lower side of a bottom element;

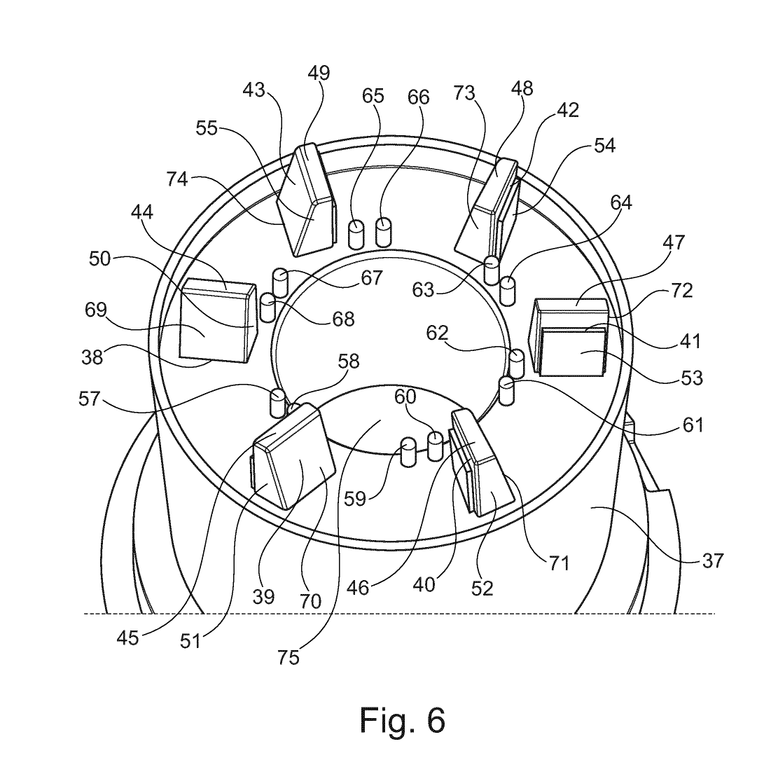

FIG. 6 the upper side of a cover element;

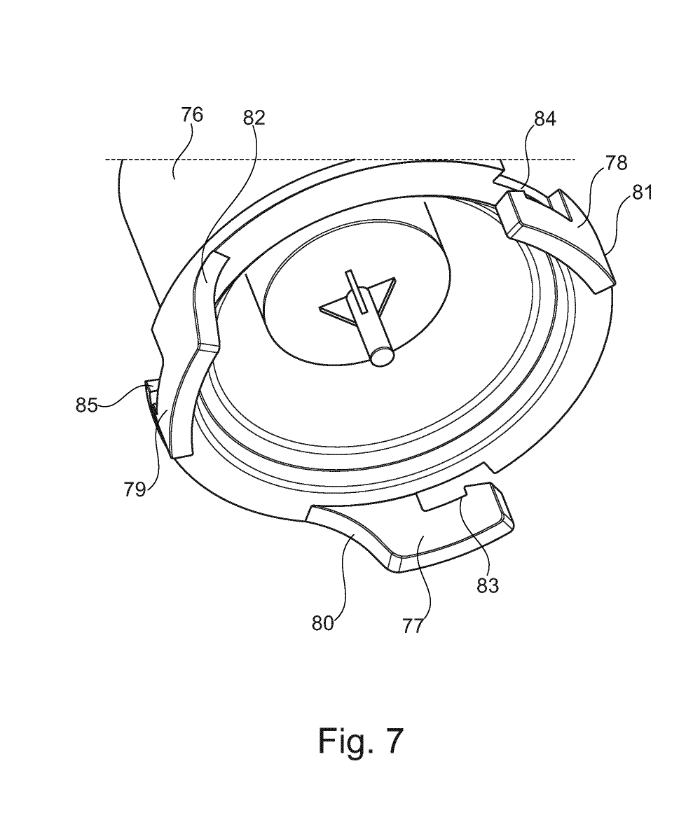

FIG. 7 the lower side of a cover element; and

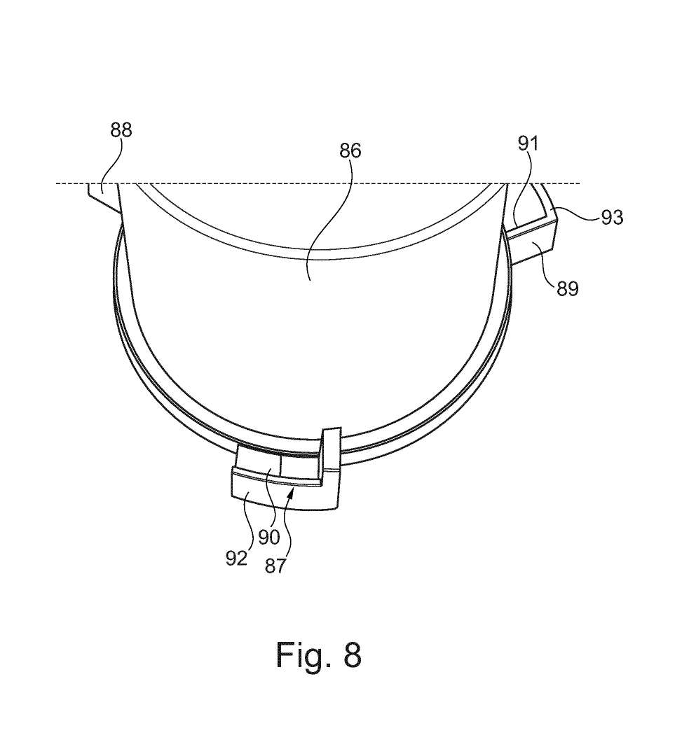

FIG. 8 a detail of a bottom element.

In FIG. 1, a bottom element 1, a cover element 2, a sealing ring 3 with an inner diameter 4 and a nozzle insert 5 is depicted. The bottom element 1, the cover element 2 and the nozzle insert 5 are formed as injection molded parts. With an injection molding method, in particular plastics can be cost-efficiently brought into the required form.

The dimensions of the nozzle insert 5, in particular of the nozzle opening 6, may have an especially large influence on the size of the aerosol particles or aerosol droplets, such that it is generally also possible to use another manufacturing method and/or material for the nozzle insert 5, with which lower manufacturing tolerances can be met. Due to the geometrically simple form of the nozzle insert 5, a change of the manufacturing method for the nozzle insert 5 does not have to lead to higher costs.

FIG. 2 shows a cover element 7, a sealing ring 8 and a bottom element 9, in which a nozzle inert 10 was inserted. The nozzle insert is fixed in a radial direction by ribs 11, 12, 13, 14, 15, 16. The ribs 11, 12, 13, 14, 15, 16 may in addition contribute to the stiffening of the bottom element 9. The nozzle insert 10 is movable in the axial direction and may be replaced with another (not shown here) nozzle insert made of another material and/or with a nozzle opening deviating from the nozzle opening 17 in the form and/or the size.

In FIG. 3, a cover element 18 and a bottom element 19 are depicted. In the bottom element 19 a nozzle insert 20 was inserted and a sealing ring 21 was slid over an outer circumferential surface 22 of the bottom element.

The sealing ring 21 can be slid over the outer circumferential surface 22 both before and after inserting the nozzle insert 20. The inner diameter of an elastic sealing ring 21 may be selected smaller than that of the outer circumferential surface 22, for example, such that after sliding over, the sealing ring 21 is held secured against loss by the outer circumferential surface 22. With a sealing ring 21 which is held secured against loss, it may be appropriate to slide it over the outer circumferential surface 22 before the nozzle insert 20 is inserted, to avoid a falling out of the nozzle insert 20 during the manipulation of the bottom element 19, for example when attaching the sealing ring 21 or in a transport of the bottom element.

FIG. 4 shows an exchangeable cartridge 23 with a bottom element 24 and a cover element 25 which are connected to each other by a bayonet catch.

In FIG. 5, the lower side of a bottom element 26 is depicted. The bottom 27 of the bottom element 26 is curved. Due to the curvature of the bottom 27, the amount of the aerosol component located in a reservoir of the bottom element, which is not outputtable, may be reduced. This may contribute to a reduction of the resources consumption and a cost reduction. The bottom element 26 is stiffened by ribs 29, 30, 31, 32, 33, 34 which originate from a cylinder barrel 28. The ribs 29, 30, 31, 32, 33, 34 may continue up to the interior of the bottom element 26 and may, corresponding to the ribs 11, 12, 13, 14, 16 shown in

FIG. 2, serve for a radially fixing of the nozzle insert. In a radial center of the bottom element 26 a hollow cylinder 35 is formed which is closed by a coverage 36. In the shown embodiment, the coverage 36 is formed integrally with the bottom 27 and the remaining elements of the bottom element 26. The hollow cylinder 35 may generally also be covered with a foil, in particular an aluminum foil or plastic foil. In an embodiment not shown here, the hollow cylinder 35 may comprise an inner thread in which a gas bottle can be screwed in.

FIG. 6 shows the upper side of a cover element 37. At the upper side of the cover element 37, opening aids 38, 39, 40, 41, 42, 43 are provided. When inserting an exchangeable cartridge which is provided with the cover element 37 into the associated device, the opening aids 38, 39, 40, 41, 42, 43 at their upper end 44, 45, 46, 47, 48, 49 firstly come into contact with a correspondingly formed contact surface of the device, and then fold around the folding lines 50, 51, 52, 53, 54, 55 before the exchangeable cartridge with the cylinder barrel 56 and the abutment pins 57 to 68 abuts onto the device. The opening aids 38, 39, 40, 41, 42, 43 at the same time rupture along the predetermined breaking locations 69, 70, 71, 72, 73, 74, such that openings to the space which is enclosed by the cover element 37 are formed, through which aerosol generated within the exchangeable cartridge can be output into the device. The hollow cylinder 75 which is formed at the upper side of the cover element 37 may at the same time serve for centering the exchangeable cartridge in the device.

In FIG. 7, the lower side of a cover element 76 is depicted. The cover element comprises three hooks 77, 78, 79 which may cooperate with corresponding protrusions of a bottom element not shown here, for forming a bayonet catch. The hooks may also be formed at the bottom element and cooperate with protrusions of the cover element. The hooks 77, 78, 79 each comprise a sliding surface 80, 81, 82, at which the corresponding protrusions of the bottom element can slide when assembling the bottom element and the cover element 76. Furthermore, at the hooks 77, 78, 79, engagement protrusions 83, 84, 85 are formed, with whose help the cover element 76 is engageable with a corresponding bottom element.

FIG. 8 shows a detail of a bottom element 86 with three protrusions 87, 88, 89 which can cooperate with hooks of a cover element for forming a bayonet catch. The protrusion 87 comprises, as well as the other protrusions 88, 89, an engagement accommodation 90, via which the bottom elements 86 is engageable with corresponding engagement protrusions of a cover element. The bottom element 86 is provided for being engaged with a corresponding cover element only once to ensure that the exchangeable cartridge can only be filled with the first aerosol component by authorized persons. In particular in medical applications of the exchangeable cartridge, it may be important that sterile conditions prevail when filling the cartridge. In addition, for medical applications typically a manipulation-proof exchangeable cartridge is required, whose medically effective first aerosol component cannot be replaced with an ineffective or maybe even harmful medium. The engagement accommodations 90, 91 are protected in the radial direction by a protection bar 92, 93. In a connected state of the bottom element 86 and the associated cover element, the engagement in this way can be protected against manipulations for opening of the exchangeable cartridge.

1-10. (canceled)

11. Exchangeable cartridge for generating an aerosol,
with at least one bottom element and
with at least one cover element,
wherein in the bottom element and/or cover element a reservoir is formed for a first, in particular liquid, aerosol component,
wherein in the exchangeable cartridge a nebulizer nozzle is formed,
wherein the bottom element and the cover element are connected to each other by a bayonet catch.

12. Exchangeable cartridge according to claim 11, wherein
the bayonet catch comprises at least one protrusion and at least one hook, and
the hook comprises a sliding surface at which the protrusion can slide when bringing together the bottom element and the cover element.

13. Exchangeable cartridge according to claim 11, wherein
the bottom element and/or the cover element comprises a centering surface.

14. Exchangeable cartridge according to claim 11, wherein
the exchangeable cartridge comprises at least one engagement protrusion assigned to the bayonet catch and at least one engagement accommodation assigned to the bayonet catch, and
the bottom element and the cover element are engageable with each other by the engagement protrusion and the engagement accommodation.

15. Exchangeable cartridge according to claim 11, wherein
the engagement protrusion and/or the engagement accommodation are formed such that the bottom element and the cover element are engageable with each other only once.

16. Exchangeable cartridge according to claim 11, wherein
the engagement protrusion and/or the engagement accommodation comprises a predetermined breaking location which, when opening the engagement, leads to a destruction, in particular a visible destruction, of the engagement protrusion and/or the engagement accommodation.

17. Exchangeable cartridge according to claim 11, wherein
the engagement protrusion and/or the engagement accommodation comprise a coverage.

18. Exchangeable cartridge according to claim 11, wherein
the exchangeable cartridge comprises a sealing ring arranged between the bottom element and the cover element.

19. Exchangeable cartridge according to claim 11, wherein
the cartridge comprises a pressure gas bottle, in particular a pressure gas cartridge, which is detachably or non-detachably connected with the bottom element and/or the cover element.

20. Exchangeable cartridge according to claim 11, wherein
the pressure gas bottle contains a fragrant and/or disinfectant and/or therapeutically effective gas, in particular oxygen.
